(19) United States
(12) Patent Application Publication
(10) Pub. No.: US 2023/0037474 A1
(43) Pub. Date: Feb. 9, 2023

(54) HEAD SIZE ADAPTATION MECHANISM FOR AN EEG NET

(71) Applicant: Magstim Group, Incorporated, Eden Prarie, MN (US)

(72) Inventors: Bernhard Gleich, Hamburg (DE); Achim Hilgers, Alsdorf (DE); Igor Berezhnoy, Eindhoven (NL); Ingmar Graesslin, Boenningstedt (DE); Ronaldus Maria Aarts, Eindhoven (NL); Raymond Van EE, Veldrop (NL)

(21) Appl. No.: 17/791,745

(22) PCT Filed: Jan. 6, 2021
(86) PCT No.: PCT/IB2021/050056

§ 371 Date: Jul. 8, 2022

Related U.S. Application Data
(60) Provisional application No. 62/959,522, filed on Jan. 10, 2020.

(51) Int. Cl. A61B 5/00; A61B 5/291; A61B 5/256; A61B 5/31

(52) U.S. Cl. CPC A61B 5/6843; A61B 5/291; A61B 5/256; A61B 5/31; A61B 5/6803; A61B 2562/046; A61B 2562/227

(57) ABSTRACT

An electroencephalography net (44) comprised of electrodes (34, 36) coupled together by a connector (28) comprising separate elastically (32) and plastically (30) deformable elements.

### FIELD OF THE INVENTION

The present invention is generally related to electroencephalography (EEG) nets, and in particular, high-density (HD) EEG nets.

### BACKGROUND OF THE INVENTION

Electroencephalography (EEG) uses a network of electrodes that are electrically coupled to the surface of the skin of a subject's scalp. The electrodes are used to measure voltages and/or currents produced by electrical activity in the brain, and a subset of the electrodes may also be used in some implementations to stimulate brain regions (via a current across the electrodes) such as for therapy or for use in electrical source imaging (ESI). A high-density (HD) EEG net comprises a high density of electrodes (e.g., over thirty-two electrodes) that are tethered together to form a cap. The HD EEG cap is used to make high resolution EEG measurements of brain activity.

Despite the superior data quality from HD EEG nets, wide spread adoption in clinical use has not yet been realized. One significant reason for this seems to be the additional cost of the HD EEG nets over conventional EEG nets (e.g., not only equipment cost but also the cost of use). For instance, one cost issue is that EEG nets for different head sizes should be at hand in the hospital. As head sizes vary considerably from infants to adults and even within groups, a multitude of nets should be in stock in the hospital. The number of sizes needed is currently somewhere between 10 and 20. As HD EEG nets become more main stream, several nets for each size in every hospital may be needed, as it is possible that patients with the same head size show up at the same time. Therefore a solution with one size for all patients or at least a configuration that minimizes the amount of nets in stock is highly desirable.

One approach to addressing the need for handling different sizes is disclosed in U.S. Patent Publication No. 2011/0077497 to Oster et al., which discloses a biomedical sensor system comprising a connector having a variable length, such that a sensor and hub connected by the connector can be positioned a variable distance apart by changing the length of the connector (see, Abstract). The biomedical sensor system is described as capable of being used in electroencephalography to monitor brain activity and diagnose brain abnormalities, among other applications (e.g., electrocardiography, electromyography, as described in paragraph [0021]), and is size-configurable to allow accommodation of a variety of subject sizes (see, paragraph) [0023]) The connector is comprised of a viscoelastic material ranging anywhere from viscoelastic materials that are largely elastic and exhibit substantial elastic deformations to viscoelastic materials that exhibit substantial plastic deformations and minimal elastic deformations (see, paragraph [0051]). Support members of the connector may be formed of a variety of materials capable of changing in length (e.g., elongating when force is applied in the direction of the elongation, e.g., see paragraph [0060]), and may be comprised of a material that changes dimensions in response to heating or cooling (e.g., heating applied to cause shrinkage, see, e.g., paragraph [0061]). These types of features lend themselves to variable sizing of a biomedical sensor system, yet different designs may be used to achieve these and/or other aims.

### SUMMARY OF THE INVENTION

One object of the present invention is to enable variable sizing of an electroencephalography (EEG) net. To better address such concerns, in a first aspect of the invention, an electroencephalography net is disclosed that is comprised of electrodes coupled together by a connector comprising separate elastically and plastically deformable elements. Through the use of elastic deformable or spring elements in cooperation with plastic deformable elements, a plurality of different net sizes may be created from a single EEG net, which provides for both flexibility in application and a reduction in quantities of EEG nets needed for stock, making high-density EEG nets more attractive in clinical settings.

In one embodiment, the EEG net comprises a first electrode; a second electrode; and a connector mechanically coupling the first and second electrodes, the connector comprising plural first elements connected to a second element separate from the plural first elements, the plural first elements comprised of an elastic deformable material, the second element comprised of a plastic deformable material. The physical arrangement and material differences of these separate connector structures provides for a variable length connector and hence flexibility for use in a plurality of different head sizes using the same EEG net.

In one embodiment, the plural first elements or the second element is comprised of a NiTiNol material. NiTiNol, also referred to as nickel titanium, is a metal alloy of nickel and titanium, and exhibits both shape memory and super elasticity, which facilitates, in conjunction with the elastic deformable material of the plural first elements, the variable length connector capability and hence size variations from a single EEG net. NiTiNol provides a further benefit in its flexibility of use. For instance, it may be used as a plastic element when the transition temperature is above room temperature and as an elastic element when the transition temperature is below room temperature.

In one embodiment, the plural first elements comprise two separate spring elements, wherein the second element is serially placed between the two separate spring elements. This physical combination of elements enables the shape deformation to a persistent dimension (e.g., upon stretching of the EEG net), and then return (e.g., upon the application of heat) to the original dimensions.

In one embodiment, the EEG net is configured as a high-density electroencephalography net. Through the reduction of needed sizes, given the ability to be deformed to a plurality of different head sizes, the use of HD EEG nets becomes more attractive from a cost and simplicity of use perspective.

In one embodiment, a system is disclosed to change the dimensions of the EEG net, including a pump; a first balloon upon which the electroencephalography net is conformally placed; and a base comprising a port, the port configured to receive the first balloon, the base operably connected to the pump, the pump configured to expand the first balloon and, correspondingly, the electroencephalography net, by filling the first balloon with a fluid. For instance, the EEG net may be expanded to fit one of a plurality of different head sizes through the use of a pump and balloon that is filled by the pump with a fluid (e.g., compressible fluid, such as air, gas, etc., or in some embodiments, using a non-compressible fluid like water).

In one embodiment, the first balloon comprises a sticky surface sufficient to cause adherence of the first and second electrodes to the first balloon. The adherence results in a uniform expansion of the EEG net while the balloon expands.

In one embodiment, the first balloon comprises indentations to respectively receive the first and second electrodes. Again, the indentations facilitate the securement of the electrodes in respective position to facilitate a uniform expansion.

In one embodiment, further comprising a second balloon, the first and second electrodes sandwiched between the first and second balloons. The arrangement of the first and second balloons provides yet another or additional mechanism to secure the electrodes in respective position uniformly as the first balloon expands against a resistance (the second balloon).

These and other aspects of the invention will be apparent from and elucidated with reference to the embodiment(s) described hereinafter.

### BRIEF DESCRIPTION OF THE DRAWINGS

Many aspects of the invention can be better understood with reference to the following drawings, which are diagrammatic. The components in the drawings are not necessarily to scale, emphasis instead being placed upon clearly illustrating the principles of the present invention. Moreover, in the drawings, like reference numerals designate corresponding parts throughout the several views.

FIG. 1 is a schematic diagram that conceptually illustrates an example high-density (HD) electroencephalography (EEG) system, in accordance with an embodiment of the invention.

FIG. 2 is a schematic diagram that illustrates an example HD EEG net in which plastic deformable elements may be inserted in respective mechanical connectors, in accordance with an embodiment of the invention.

FIG. 3 is a schematic diagram that conceptually illustrates an example mechanical connector arrangement with plural elastic deformation elements and a plastic deformation element, in accordance with an embodiment of the invention.

FIGS. 4A-4C are schematic diagrams that illustrates an example system for adjusting a size of an HD EEG net, in accordance with an embodiment of the invention.

FIG. 5 is a flow diagram that illustrates an example method for adjusting a size of an HD EEG net, in accordance with an embodiment of the invention.

### DETAILED DESCRIPTION OF EMBODIMENTS

Disclosed herein are certain embodiments of an electroencephalography (EEG) net and associated systems and methods that make use of elastic and plastic deformable structures in the net EEG that enable a single EEG net to be expanded to one of a plurality of different head sizes. The principles set forth herein for the EEG net structures has particularly beneficial aspects for high-density (HD) EEG nets, which has experienced slower acceptance in the market when compared to regular EEG nets. In one embodiment, the connectors mechanically connecting plural adjacent electrodes together are comprised of plural first elements (referred to also herein as spring elements) connected to a second element (referred to herein as a plastic element, though plastic referring to the deformation property and not necessarily always a plastic material) separate from the plural first elements, the plural first elements comprised of an elastic deformable material, the second element comprised of a plastic deformable material. Through this arrangement of structures of the connection, a single EEG net may be adapted for many head sizes and hence a reduction in the number of EEG nets to stock in a clinical setting is realized. Certain system and method embodiments for expanding the EEG net are also described herein.

Digressing briefly, hospitals and other clinical settings that perform EEG testing of subjects have to stock different EEG nets having different EEG sizes to accommodate the many varied head sizes encountered today (e.g., adults, children, etc.), which particularly affects acceptance of the higher performing, higher cost HD EEG nets. In certain embodiments, an EEG net is disclosed that comprises a physical arrangement or structure of the mechanical connectors (that couple the electrodes of the net together and provide flexibility in the fitting of the EEG net) that includes a serial arrangement of elastic and plastic deformable elements, thus providing for ease of fitting and reduction in stocks of needed EEG nets, which when applied to HD EEG, may engender more widespread acceptance of HD EEG nets.

Having summarized certain features of an EEG net of the present disclosure, reference will now be made in detail to the description of an EEG net as illustrated in the drawings. While an EEG net will be described in connection with these drawings, there is no intent to limit it to the embodiment or embodiments disclosed herein. For instance, emphasis below is on HD EEG nets (e.g., having over thirty two electrodes and at this time including up to 256 electrodes), though it should be appreciated that smaller nets may similarly benefit and hence are contemplated to be within the scope of the disclosure. Also, though the connectors for mechanically connecting (e.g., and not for electrical/electronic signaling, where a separate cabling connector is disclosed for that purpose) electrodes into a net or cap are disclosed as including the spring and plastic elements, in some embodiments, the same or similar structure may be used for a connector that serves both the mechanical and electrical connectivity for the EEG net in some embodiments (e.g., by embedding a conductive coil or wire in the mechanical connector). Further, although the description identifies or describes specifics of one or more embodiments, such specifics are not necessarily part of every embodiment, nor are all of any various stated advantages necessarily associated with a single embodiment. On the contrary, the intent is to cover all alternatives, modifications and equivalents included within the principles and scope of the disclosure as defined by the appended claims. For instance, two or more embodiments may be interchanged or combined in any combination. Further, it should be appreciated in the context of the present disclosure that the claims are not necessarily limited to the particular embodiments set out in the description.

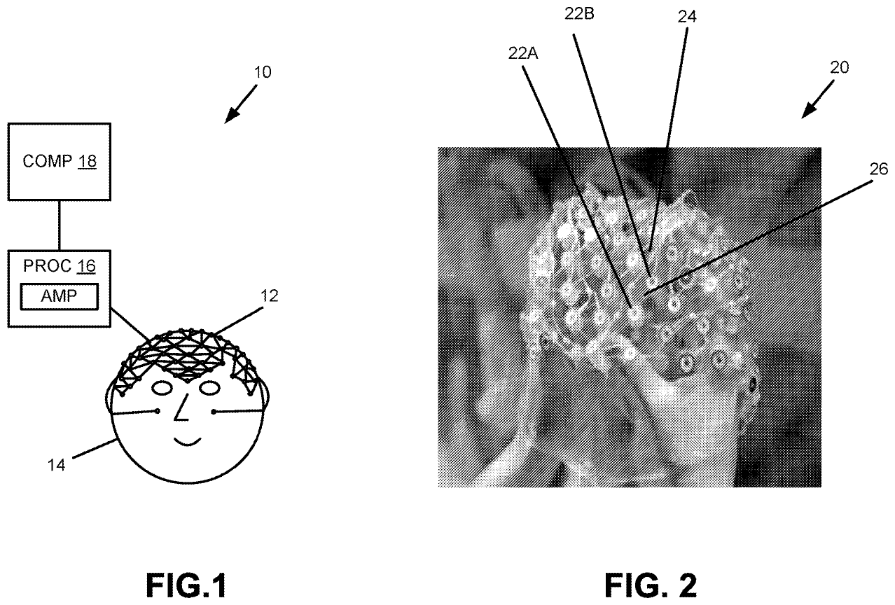

FIG. 1 illustrates an example HD EEG system 10 that is used to measure voltages and/or currents produced by electrical activity in the brain and optionally stimulate (via current source circuitry) deep brain regions and/or cortical surfaces of the brain (e.g., for electrical source imaging or ESI). The HD EEG system 10 includes an HD EEG net 12 that is fitted onto a scalp surface of a subject 14. The HD EEG net 12 comprises a plurality of electrodes. The HD EEG net 12 may include 64, 128, or 256 EEG electrodes and hence EEG electrode channels. In some embodiments, other EEG nets may be used with fewer electrodes (and hence channels), including 32 electrodes. The HD EEG net 12 is coupled to a processing device 16. The processing device 16 comprises an amplifier that is configured to filter, measure, and sample the EEG signals acquired by the HD EEG net 12, and then transfer the digitized samples to a controller/computing device 18 for further processing and/or display. Note that in some embodiments, the HD EEG system 10 may cooperate with other measurement modalities, including a magnetic resonance imaging system (e.g., for electrical source imaging). For instance, in electrical source imaging, data from a subset of the electrodes (e.g., sensing electrodes) of the HD EEG net 12 may be used to localize the seizure focus on the cortical surface and some electrodes may be used for stimulation of brain regions. In other words, signals to/from the HD EEG electrodes of the HD EEG net 12 are used to record brain activity of the subject 14 from the cortical regions of the brain. The controller/computing device 18 may be coupled to a display screen and/or storage device or system for display and/or recording of sensed and/or other data. As the applications for an HD EEG system are known, further description of the same is omitted here for brevity.

FIG. 2 illustrates an example HD EEG net 20 in which plastic elements may be inserted in respective mechanical connectors. The HD EEG net 20 may be similar to that shown for the HD EEG net 12 of FIG. 1, and includes plural electrodes 22 (e.g., 22A, 22B, etc.) that are electrically connected via an electrical connector 24 (e.g., insulated cabling wire) and connected via a mechanical connector 26 (shown as somewhat translucent connector strips between electrodes 22) that in one embodiment comprises spring elements coupled to a plastic element, as described below. In one embodiment, there are a plurality of electrical connectors 24 and mechanical connectors 26, with each of the connectors 24, 26 coupled to two or more adjacent electrodes 22. Note that the electrical connector 24 also serves a mechanical coupling function (yet absent the combination of plastic and elastic components) among two or more adjacent electrodes 22, whereas in contrast, the mechanical connector 26, at least in one embodiment, does not carry (or have embedded therein) signaling wire (unlike the electrical connector 24). The mechanical connectors 26 facilitate the stretching of the HD EEG net 20 to enable conformal fitting upon a subject's head. As explained further below, the mechanical connectors 26 comprise spring elements serially coupled to a plastic element to enable the HD EEG net 20 to be adapted in size to fit a plurality of head sizes.

In general, certain embodiments of an HD EEG net according to the description herein insert elements into the mechanical connector 26 that may be stretched. Pure rubber bands are not considered practical since use of such elements may lead to excessive forces for large heads. Instead of using only elastic material, certain embodiments of an HD EEG net insert spring elements along with one or more elements comprising plastic properties in each mechanical connector 26. Accordingly, when each mechanical connector 26 that flexibly joins two or more adjacent electrodes is stretched, each connector 26 does not return to the original size afterwards. After use, the plastic elements are heated (e.g., merely as a side effect during cleaning) and shrink to their original dimensions. For instance, the elastic elements are soft until a certain elongation is reached. Afterwards, the elastic element gets considerably harder. This feature can be easily achieved by incorporating some fibers (e.g., commercially available) in a rubber band. Once the rubber has stretched to the point where the fibers are fully stretched (e.g., no more bends), the fibers reach a stage or phase where they become load bearing elements. When this stage or phase is reached, the plastic elements can be subjected to large forces and stretched. In the context of a system that utilizes a balloon to adjust the size of the HD EEG net (explained further in association with FIGS. 4A-4C), the balloon diameter for the stretching is larger than a target head diameter. This means the final result is a net, which has already approximately the desired size and is easy to apply to the patient (does not collapse, so a very small size). The forces on the head may be adjusted by choosing the right rubber material, and this is largely independent from the plastic material. In some embodiments, a further improvement may be realized using a structure that avoids a further collapse of the rubber band (e.g., the band is inside a housing with a lid attached to the rubber band). This means there is already an initial force needed to stretch the rubber band and the force in the net remains largely independent of the stretching distance when being between the minimum (lid on case) and maximum (elongated fibers) distance. This feature may improve patient comfort.

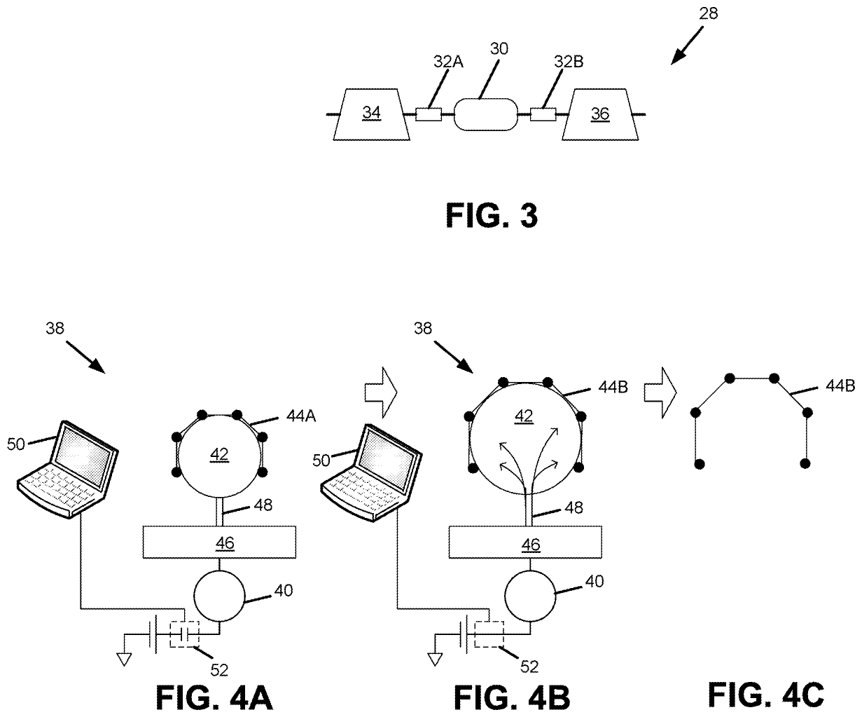

Referring now to FIG. 3, shown is a conceptual illustration of an embodiment of a mechanical connector 28 with spring elements and a plastic element. The mechanical connector 28 may mechanically couple two or more electrodes together, as similarly shown in FIG. 2 for mechanical connector 26. In one embodiment, the mechanical connector 28 comprises a plastic element 30 disposed between plural (e.g., two, though not limited to two) spring elements 32 (e.g., 32A, 32B), the mechanical connector 28 mechanically coupling two electrodes 34, 36 together. In other words, the mechanical connector 28 comprises a serial arrangement of an electrode 34, a spring element 32A, the plastic element 30, the spring element 32B, and the electrode 36. In some embodiments, one or more components of the mechanical connector 28 may be replaceable. In some embodiments, the arrangement of these elements may be configured differently, including using a different quantity and/or order of the spring and/or plastic elements 32, 30, respectively. The spring elements 32 are comprised of an elastic deformable material, which may include an elastomer. The plastic element 30 is comprised of a plastic deformable material, which may include a nickel titanium (NiTiNol) coil, a cross-linked polymer (e.g., similar to that used in shrinkable hoses), among other materials comprising plastic deformable properties. In some embodiments, the NiTiNol material may provide for elastic properties under some conditions and plastic properties under other conditions. For instance, it may be used as a plastic element when the transition temperature is above room temperature and as an elastic element when the transition temperature is below room temperature. When the plastic element 30 comprises a metallic material, there are multiple benefits, including improved durability and the capability of the connectors to be routed along the coiled wire structure to prevent or mitigate the risk that the cables are entangled in a pre-expanded or low expansion configuration.

Manufacturing of the mechanical connector 28 may be achieved according to automated, semi-automated, or manual techniques, including through the use of injection molding, manual fabrication (e.g., in the case where metallic coils are used). The joining of the elements may be performed in many ways. For instance, there may be hooks at the end of the elements and they are connected during the manufacturing process, wherein the hooks may be crimped. There may be a wires coming from the elements that are crimped or soldered together. The elements may be all placed in a molding tool together with the rest of the net components and they may be joined by a molding process, etc. The methods may be combined which is especially useful, if there are electrical connectors going through the elements, which may be soldered or crimped, with the whole assembly finally covered by an elastic resin.

FIGS. 4A-B are schematic diagrams that illustrates an example system 38 for adjusting a size of an HD EEG net, with FIG. 4C showing the result of the expansion. It should be appreciated by one having ordinary skill in the art that the system 38 depicted in FIGS. 4A-4C is one example embodiment, and that other mechanisms for expanding an HD EEG net may be used. In one embodiment, the system 38 comprises a pump 40, a balloon 42 upon which an HD EEG net 44 is conformally or substantially conformally placed, and a base 46 comprising a port 48 (e.g., at one end, comprising a shrader connection, quick-connect plugs, push-to-connect NPT fittings, check-valve, etc.), the port configured to receive and secure the balloon 42 (e.g., compression fit, etc.), the base 46 operably coupled to the pump 40 (e.g., via a hose or tubing connection). Any arrangement may be used to couple the balloon 42 to the pump 40, including a simple a clamp ring, which may be used to couple the pump 40 to the stem of the balloon 42. The pump 40 may comprise an A.C. (alternating current) or D.C. (direct current) pump, or pneumatic or other types of pumps in some embodiments. In some embodiments, the pump 40 may be configured similarly to off-the-shelf electric pumps used to inflate blow-up beds, rafts, etc. In some embodiments, the pump 40 may be similarly configured to off-the-shelf pumps that are used to inflate bicycle tires or inflatable balls (e.g., basketballs, beach-balls, etc.) manually. In effect, whether manually or automatically operated, the pump 40 is configured to discharge fluid into the balloon 42 via the port 48, resulting in a corresponding expansion of the balloon 42 and hence the HD EEG net 44 positioned upon the balloon 42. The balloon 42 comprises, in one embodiment, a thick-walled balloon 42 of rubber, latex, or other known materials that permit the balloon 42 to expand when being filled with fluid and return back to its original shape and dimensions when the fluid is withdrawn. In some embodiments, a single-size, round balloon 42 may suffice for all applications, though in some embodiments, additional sized balloons 42 may be available to choose from depending on the application. The fluid may be a compressible fluid, including air and/or gas, or in some embodiments, may be an incompressible fluid (e.g., water) that is discharged from the pump 40 through the port 48 and into the interior of the balloon 42. The base 46 may comprise tubing, channels, or other passageways that enable the flow of the fluid (e.g., the pump 40 is fluidly coupled to the port 48). Note that the balloon 42 may be semi-inflated to receive and support the HD EEG net 44 in its original, un-stretched condition. In some embodiments, the balloon 42 may have a sufficient structure to retain a shape, in its unexpanded condition, that is suitable to receive and support the HD EEG net 44 in its un-expanded condition.

The HD EEG net 44 is conformally placed onto the external surface of the balloon 42. In one embodiment, to ensure a uniform elongation of the plastic elements of the mechanical connectors of the HD EEG net 44, the electrodes should stick with sufficient adhesive force on the balloon 42 to remain in place before and during expansion. Accordingly, in one embodiment, the balloon 42 comprises a sticky exterior surface that suitably secures the balloon 42 to the electrodes of the EEG net 44 throughout the size adjustment process. In some embodiments, the exterior of the balloon 42 comprises appropriate indentations for each electrode of the HD EEG net 44 in the balloon hull to accommodate each electrode in addition to or in lieu of the sticky surface. In some embodiments (e.g., in addition to one or more of the sticky surface, indentations or in lieu of the same), a second balloon (not shown) may be placed over the HD EEG net 44 to press the electrodes against the first balloon 42 (e.g., provide increasing resistance) and ensure fixed electrode positions during the size adjustment process. In the case of double-shelled balloons 42, the outer shell may serve as a shield in the case of rupture of the inner shell.

In one embodiment, the system 38 may further comprise a control system comprising a control unit/computing device 50 and a switching mechanism 52 (e.g., relay(s), contactor(s), transistor(s), and/or logic devices, etc.) that controls power to the pump 40, and accordingly, is switched on and off (e.g., and/or modulated) to control the adjustment in size of the balloon 42 (and hence the adjustment in size of the HD EEG net 44 that resides on the balloon 42). In one embodiment, an operator, technician, medical assistant, or other personnel may enter a head size of a subject (or in some embodiments, a net size, such as where the subject is re-visiting) into the control unit 50. For instance, the desired head size may be typed in at the control unit 50, and the control units 50 cooperates with the switching mechanism 52 to cause the pump 40 to adjust its speed of inflation automatically (e.g., until the determined size or fault condition is reached). For instance, circuitry in the control unit (e.g., a combination of logic gates using timers/counters) 50 may translate the target size dimension into a duration and/or rate of inflation, and/or sensors may be positioned proximal to the balloon 42 to enable feedback of whether the appropriate size dimension (or fault condition) has been reached. In one embodiment, balloon size detection may be achieved through the use of an assembly of two plates with force sensors (e.g., simple switches). The distance of the plates may be adjustable. When a force is detected, inflation stops. In some embodiments, fluid pressure may be monitored, such that when fluid pressure reaches a predetermined pressure value, inflation stops. Other measures may control inflation (or termination of inflation), including the use of the slope (e.g., as the pressure increases and then decreases during the inflation process). In some implementations, the pump 40 may need to inflate and then deflate a little to facilitate easy removal of the HD EEG net 44 from the balloon. In some embodiments, control may be achieved by personnel operating a manual switch to regulate the flow, with termination of the expansion when sensors provide feedback through visual, audible, and/or tactile mechanisms alerting personnel to stop the expansion, or based on observation (e.g., a head-size scale may be superimposed in the view of the technician, which the technician watches for alignment of the balloon expansion to the appropriate head size scale to enable a determination of when to terminate the expansion of the balloon 42). These and/or other mechanisms may be used to enable manual, automatic, or semi-automatic HD EEG size adjustment.

Execution of the control unit 50 may be implemented by one or more processors under the management and/or control of an operating system. In some embodiments, a more rudimentary form of control may be implemented (e.g., without an operating system). Processing of the control unit 50 may be achieved using a custom-made or commercially available processor, including a single or multi-core central processing unit (CPU), graphics processing unit (CPU), or an auxiliary processor among several processors, a semiconductor based microprocessor (in the form of a microchip), a macroprocessor, one or more application specific integrated circuits (ASICs), field programmable gate arrays (FPGUs), a plurality of suitably configured digital logic gates, programmable logic controller (PLC), and/or other known electrical configurations comprising discrete elements both individually and in various combinations to coordinate the overall operation of the control unit 50. The control unit 50 may comprise a memory, which may include any one or a combination of volatile memory elements (e.g., random-access memory RAM, such as DRAM, and SRAM, etc.) and nonvolatile memory elements (e.g., ROM, Flash, solid state, EPROM, EEPROM, hard drive, CDROM, etc.). The functionality for enabling operation of the pump 40 to cause an adjustment in the size of the HD EEG net may include software (including firmware, middleware, microcode, etc.) that, when implemented by one or more processors of the control unit 50, causes the processor(s) to enable pump operation via the switching mechanism 52 or otherwise. In some embodiments, the control and switching mechanism may reside in a single unit. The software may be stored on a variety of non-transitory computer-readable (storage) medium for use by, or in connection with, a variety of computer-related systems or methods. In the context of this document, a computer-readable medium may comprise an electronic, magnetic, optical, or other physical device or apparatus that may contain or store a computer program (e.g., executable code or instructions) for use by or in connection with a computer-related system or method. The software may be embedded in a variety of computer-readable mediums for use by, or in connection with, an instruction execution system, apparatus, or device, such as a computer-based system, processor-containing system, or other system that can fetch the instructions from the instruction execution system, apparatus, or device and execute the instructions.

In some embodiments, size adjustment functionality may be implemented without the use of software. For instance, a sensor (e.g., load sensor) may be used that senses the placement of the HD EEG net onto the balloon, which when activated, causes the pump to be energized via an intermediate relay or switch. Cessation of the filling may be triggered via a sensor and/or manual observation as expressed above.

As to one example operation, as shown in FIG. 4A, the HD EEG net 44A is conformally placed over the balloon 42, the HD EEG net 44A in its resting, un-stretched configuration. The control unit 50 causes (e.g., upon user input, or based on sensing the HD EEG net 44 on the balloon 42) the switching mechanism 52 to change its state, resulting in the pump 40 being energized and discharging fluid into the balloon 42. To ensure a uniform stretching of the elements of the HD EEG net 44, the HD EEG net 44 is expanded through inflation of the balloon 42, resulting in the expansion of the HD EEG net 44A to its targeted head dimension 44B as shown in FIG. 4B. The result of the adjustment process is an HD EEG net 44B (FIG. 4C) that is now suitable for a head size that is different from its original shape and dimensions. In other words, the HD EEG net 44, having plural mechanical connectors that have elastic deformable properties, is capable of being expanded to any one of a plurality of different head sizes and retains that targeted shape/dimension due to the plastic deformable properties of the mechanical connectors of the HD EEG net 44. The original size/dimensions may be achieved through heating of the HD EEG nets (e.g., which may be incidental to a cleaning/sterilization process or as part of a shrinkage protocol).

Note that in some embodiments, other mechanisms may be used to expand the HD EEG nets to different head sizes. For instance, the balloons 42 may be expanded manually using a manually operated pump. In some embodiments, the pump 40 and/or control system (e.g., control unit 50, switching mechanism 52) may be integral to the base 46. In some embodiments, the balloon 42 may be replaced with a series of 3D-printed head models of different head sizes over which the HD EEG net is expanded manually, which enables the creation of personalized head models that may be used to adapt for standard and non-standard head shapes.

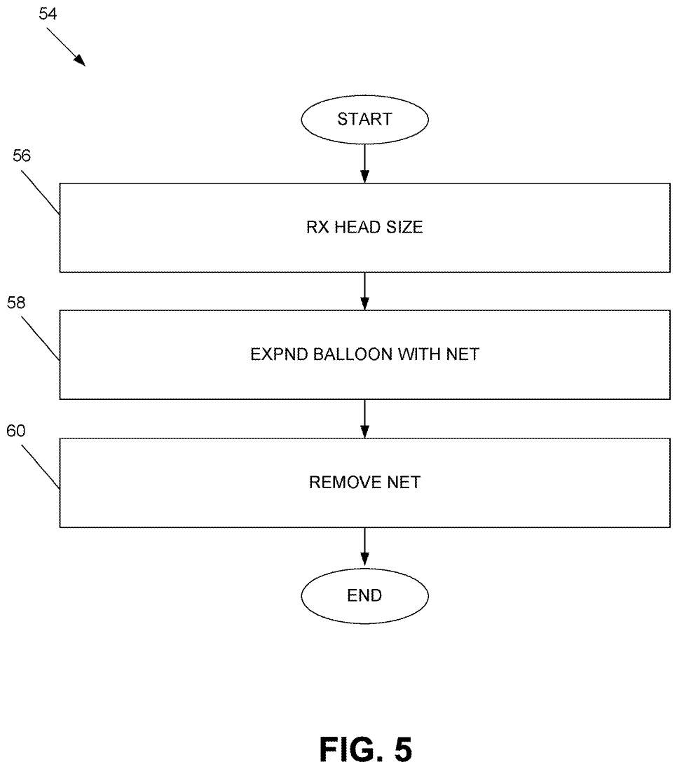

Having described certain embodiments of an example HD EEG system and corresponding apparatus and system for adjusting the size of the HD EEG net, it should be appreciated that an example EEG net size adjustment method, depicted in FIG. 5 and denoted as method 54, which is shown bounded by a start and end, comprises receiving a size dimension corresponding to an EEG net (56), expanding the EEG net by causing an expansion of a balloon upon which the EEG net is placed (58), and removing the EEG net from the balloon (60). With the removal of the EEG net, the plastic elements of the mechanical connectors of the EEG net enable the expanded shape to be retained for use on a subject's head having the corresponding head size dimension.

Any process descriptions or blocks in flow diagrams should be understood as representing steps, modules, segments, or portions of code which include one or more executable instructions for implementing specific logical functions or steps in the process, and alternate implementations are included within the scope of the embodiments in which functions may be executed out of order from that shown or discussed, including substantially concurrently or in reverse order, depending on the functionality involved, as would be understood by those reasonably skilled in the art of the present disclosure. In some embodiments, one or more steps may be omitted, or further steps may be added.

In one embodiment, an electroencephalography net is disclosed, comprising: a first electrode; a second electrode; and a connector mechanically coupling the first and second electrodes, the connector comprising plural first elements connected to a second element separate from the plural first elements, the plural first elements comprised of an elastic deformable material, the second element comprised of a plastic deformable material.

In one embodiment, the preceding electroencephalography net, wherein each of the plural first elements comprises an elastomer.

In one embodiment, any one of the preceding electroencephalography nets, wherein the second element comprises a cross-linked polymer or a metal.

In one embodiment, any one of the preceding electroencephalography nets, wherein the plural first elements or the second element is comprised of a NiTiNol material.

In one embodiment, any one of the preceding electroencephalography nets, wherein the plural first elements comprise two separate spring elements, wherein the second element is serially placed between the two separate spring elements.

In one embodiment, any one of the preceding electroencephalography nets, further comprising a plurality of additional electrodes with adjacent electrodes among each of the plurality of additional electrodes mechanically coupled to each other via a respective connector comprising the first and second elements.

In one embodiment, any one of the preceding electroencephalography nets, further comprising a second connector electrically coupling the first and second electrodes.

In one embodiment, any one of the preceding electroencephalography nets, further comprising an amplifier electrically coupled to the second connector.

In one embodiment, any one of the preceding electroencephalography nets further configured as a high-density electroencephalography net.

In one embodiment, a system for adjusting a size of any one of the preceding electroencephalography nets is disclosed, comprising: a pump; a first balloon upon which the electroencephalography net is conformally placed; and a base comprising a port, the port configured to receive the first balloon, the base operably connected to the pump, the pump configured to expand the first balloon and, correspondingly, the electroencephalography net, by filling the first balloon with a fluid.

In one embodiment, the preceding system, further comprising a control unit configured to receive one of a head size or a target electroencephalography net dimension and enable the pump to expand the first balloon according to the one of the head size or the electroencephalography net dimension.

In one embodiment, any one of the preceding systems, wherein the first balloon comprises a sticky surface sufficient to cause adherence of the first and second electrodes to the first balloon.

In one embodiment, any one of the preceding systems, wherein the first balloon comprises indentations to respectively receive the first and second electrodes.

In one embodiment, any one of the preceding systems, further comprising a second balloon, the first and second electrodes sandwiched between the first and second balloons.

In one embodiment, a method for adjusting the size of the electroencephalography net according to any one of the preceding systems is disclosed.

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims. Note that various combinations of the disclosed embodiments may be used, and hence reference to an embodiment or one embodiment is not meant to exclude features from that embodiment from use with features from other embodiments. In the claims, the word “comprising” does not exclude other elements or steps, and the indefinite article “a” or “an” does not exclude a plurality. A single processor or other unit may fulfill the functions of several items recited in the claims. The mere fact that certain measures are recited in mutually different dependent claims does not indicate that a combination of these measures cannot be used to advantage. A computer program may be stored/distributed on a suitable medium, such as an optical medium or solid-state medium supplied together with or as part of other hardware, but may also be distributed in other forms. Any reference signs in the claims should be not construed as limiting the scope.

1. An electroencephalography net, comprising:
a first electrode;
a second electrode; and
a connector mechanically coupling the first and second electrodes, the connector comprising plural first elements connected to a second element separate from the plural first elements, the plural first elements comprised of an elastic deformable material, the second element comprised of a plastic deformable material.

2. The electroencephalography net of claim 1, wherein each of the plural first elements comprises an elastomer.

3. The electroencephalography net of claim 1, wherein the second element comprises a cross-linked polymer or a metal.

4. The electroencephalography net of claim 1, wherein the plural first elements or the second element is comprised of a NiTiNol material.

5. The electroencephalography net of claim 1, wherein the plural first elements comprise two separate spring elements, wherein the second element is serially placed between the two separate spring elements.

6. The electroencephalography net of claim 1, further comprising a plurality of additional electrodes with adjacent electrodes among each of the plurality of additional electrodes mechanically coupled to each other via a respective connector comprising the first and second elements.

7. The electroencephalography net of claim 1, further comprising a second connector electrically coupling the first and second electrodes.

8. The electroencephalography net of claim 1, further comprising an amplifier electrically coupled to the second connector.

9. The electroencephalography net of claim 1 further configured as a high-density electroencephalography net.

10. A system for adjusting a size of the electroencephalography net of claim 1, comprising:
a pump;
a first balloon upon which the electroencephalography net is conformally placed; and
a base comprising a port, the port configured to receive the first balloon the base operably connected to the pump, the pump configured to expand the first balloon and, correspondingly, the electroencephalography net, by filling the first balloon with a fluid.

11. The system of claim 1, further comprising a control unit configured to receive one of a head size or a target electroencephalography net dimension and enable the pump to expand the first balloon according to the one of the head size or the electroencephalography net dimension.

12. The system of claim 1, wherein the first balloon comprises a sticky surface sufficient to cause adherence of the first and second electrodes to the first balloon.

13. The system of claim 1, wherein the first balloon comprises indentations to respectively receive the first and second electrodes.

14. The system of claim 1, further comprising a second balloon, the first and second electrodes sandwiched between the first and second balloons.

15. A method comprising:
providing an electroencephalography net, the electroencephalography net comprising a pump; a first balloon upon which the electroencephalography net is conformally placed; and a base comprising a port, the port configured to receive the first balloon the base operably connected to the pump, the pump configured to expand the first balloon and, correspondingly, the electroencephalography net, by filling the first balloon with a fluid; and
pumping the fluid into the first balloon to expand the first balloon.

16. A non-transitory computer-readable medium comprising instructions that, when executed by one or more processors, causes the one or more processors to:
receiving a size dimension corresponding to an electroencephalography net, the electroencephalography net comprising a pump; a first balloon upon which the electroencephalography net is conformally placed; and a base comprising a port, the port configured to receive the first balloon the base operably connected to the pump, the pump configured to expand the first balloon and, correspondingly, the electroencephalography net, by filling the first balloon with a fluid; and
causing the pump to pump fluid into the first balloon based on the size dimension.
